(19) United States
(12) Patent Application Publication
(10) Pub. No.: US 2008/0103540 A1
(43) Pub. Date: May 1, 2008

(54) System and Method for Arrhythmia Discrimination with Atrial-Ventricular Dissociation

(76) Inventor: Jian Cao, Shoreview, MN (US)

Correspondence Address: MEDTRONIC, INC., 710 MEDTRONIC PARKWAY NE, MINNEAPOLIS, MN, 55432-9924, US

(21) Appl. No.: 11/554,276

(22) Filed: Oct. 30, 2006

(51) Int. Cl. A61N 1/00

(52) U.S. Cl. 607/9

(57) ABSTRACT

Ventricular arrhythmias are monitored and classified based upon various criteria. One criterion is PR dissociation which is determined by comparing the PR interval in a current RR cycle to a median PR value. If the current PR interval varies too far from the median, then dissociation is indicated. Often, there are multiple atrial events in a given RR interval and the PR value has been based upon the last atrial event in the cycle. Dual buffers are provided to track a last PR value for a given interval as well as a penultimate PR interval and association is determined if either value is within an acceptable variance of a median PR value.

### BACKGROUND

The present invention relates generally to implantable medical devices (IMDs) and more specifically to implantable cardioverter defibrillators (ICDs) that sense cardiac parameters and are capable of delivering a high energy therapy to cardiovert or defibrillate a heart.

### DESCRIPTION OF THE RELATED ART

ICDs are well known, cost effective, life saving medical devices that provide various cardiac therapies including pacing, cardioversion and/or defibrillation. Pacing is most commonly provided for bradycardia; however, there are numerous pacing therapies useful with tachycardia such as overdrive pacing and anti-tachy pacing (ATP). Defibrillation delivers a high energy waveform across a portion of the heart, often on the order of 20-40 joules. Defibrillation interrupts a chaotic rhythm, such as for example, atrial or ventricular fibrillation, and allows a normal sinus rhythm to emerge intrinsically or via subsequent pacing.

As indicated, tachycardia may originate from the atria, the ventricles or both simultaneously (double tachycardia). An arrhythmia in either the ventricles or the atria may affect the other. That is, an abnormally fast atrial rhythm may conduct to the ventricles causing a correspondingly high ventricular rate. Conversely, an abnormally fast ventricular arrhythmia may conduct retrograde into the atria. Thus, while it is generally easy to identify that some type of arrhythmia is occurring, there is sometimes a challenge in identifying its true nature and origin.

As a generalization, ventricular tachycardia is more serious and often requires more immediate therapy than atrial tachycardia. That is, sinus tachycardia, atrial fibrillation (AF), and atrial flutter (AFL) may be tolerated whereas ventricular tachycardia (VT), fast ventricular tachycardia (FVT), and ventricular fibrillation (VF) may immediately lead to significant hemodynamic compromise or sudden cardiac arrest (SCA) depending upon the specific condition, rate, etc.

The general distinction between the immediate affect of atrial versus ventricular tachycardia as well as the gradation of seriousness in ventricular tachycardia provide for several therapy options. First, in some instances therapy may be withheld and the condition may self correct or may be tolerated by the patient. For example, AF may spontaneously terminate. Second, less aggressive therapies may be provided such as overdrive pacing, ATP, or drug titration. Some of these less aggressive options may be appropriate in atrial tachycardia and with some VT, provided the rate is below a threshold. Finally, the most aggressive therapy is the delivery of a high energy defibrillation shock, which is highly effective. It should also be appreciated that prior to delivering defibrillation, ATP may be provided while the ICD prepares. The ATP may terminate the arrhythmia in some cases before the device actually delivers defibrillation, which then inhibits that delivery.

The basic guiding principle for defibrillation is to deliver the therapy whenever necessary, withhold when not necessary, and deliver if uncertain. As indicated, some events such as AF may self terminate or other therapies terminate the AF and many IMDs provide patients with the ability to elect or withhold defibrillation for AF and similar arrhythmias. This is important in that defibrillation therapy can be uncomfortable. Further, from a physical and psychological perspective there may be patient frustration in receiving defibrillation therapy for misclassified events. From a device perspective, the delivery of unnecessary therapy depletes the internal power supply.

Thus, modern ICDs need to provide therapy delivery capabilities but must also provide accurate and reliable sensing and arrhythmia discrimination capabilities. The various device manufacturers have algorithms to interpret the sensed cardiac data and take appropriate actions. These algorithms are improved, varied and modified as new therapies, diagnostic capabilities, or sensory inputs are provided. There is also a basic underlying set of rules that form the core of the discrimination algorithm. One example is referred to as PR Logic™, as used by Medtronic, Inc. in many of their ICDs. Certain embodiments of this algorithm are described in U.S. Pat. Nos. 6,487,443; 6,259,947; 6,141,581; 5,855,593; and 5,545,186 each to Olson et al. and assigned to Medtronic, Inc and all are herein incorporated by reference in their entireties.

### BRIEF DESCRIPTION OF THE DRAWINGS

FIG. 1 is a block diagram of an implantable cardiac defibrillator (ICD).

FIG. 2 is a flowchart of a process for broadly classifying tachycardia.

FIG. 3 is a flowchart describing one process for determining PR dissociation.

FIG. 4 is a timing diagram illustrating an R-R interval with multiple atrial events.

FIG. 5 is a flowchart describing a process for determining a PR interval within an R-R interval.

FIG. 6 is a timing diagram that graphically illustrates certain concepts of the process from FIG. 5.

### DETAILED DESCRIPTION

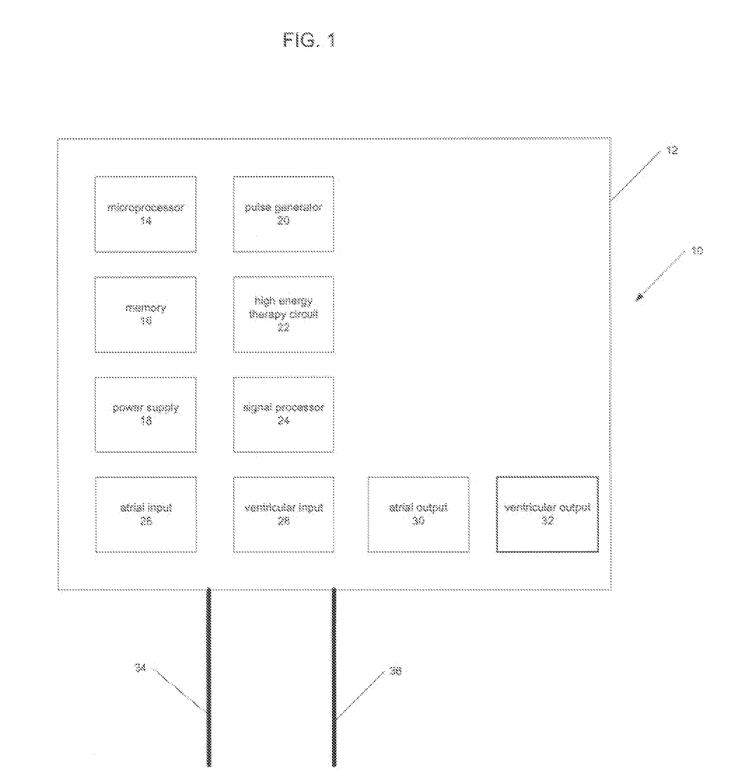

FIG. 1 is a block diagram of an ICD 10 and the various operably interconnected components and modules. ICD 10 includes a housing 12 containing a microprocessor 14, memory 16, and a power supply 18, such as a battery. Also included is a pulse generator 20, a high energy therapy circuit 22 for providing defibrillation, a signal processor 24 to process data received via an atrial input 26 and a ventricular input 28 according to algorithms retained in memory 16, encoded into firmware or otherwise provided. An atrial output module 30 and ventricular output module 32 are provided to control delivered therapies, typically via atrial lead(s) 34 and ventricular lead(s) 36. FIG. 1 is not meant to illustrate each component of an ICD as this is generally well known in the art. It should be appreciated that the present invention may be utilized in a classic ICD having a housing implanted subcutaneously or submuscularly with lead(s) extending into the heart transvenously or extending to an epicardial portion or the heart. Alternatively, the present invention may be utilized in an implantable subcutaneous defibrillator that is without having lead transvenously implanted into or onto the heart.

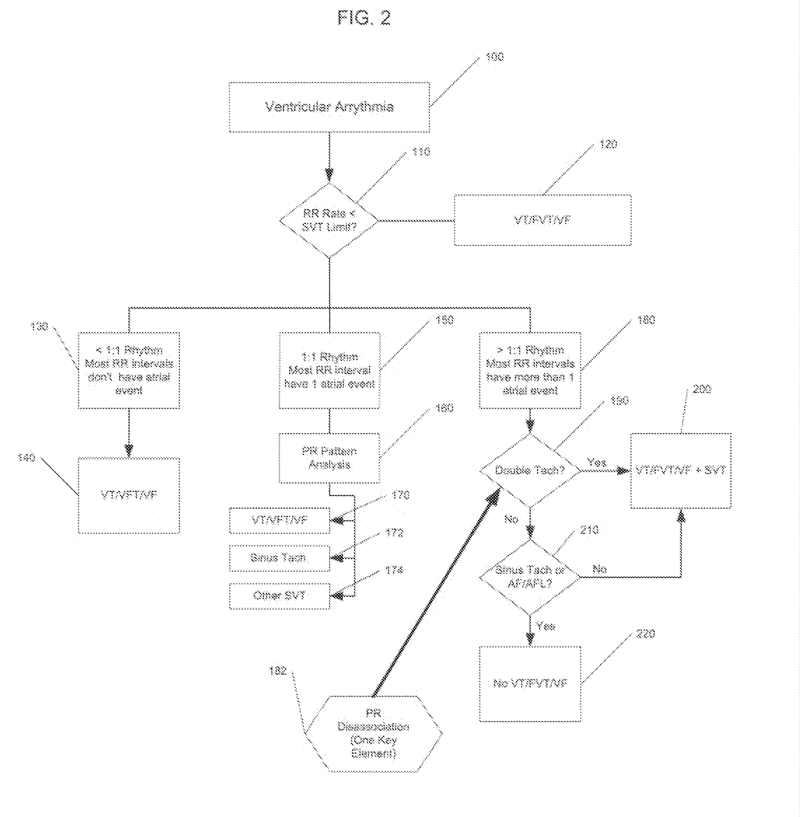

FIG. 2 is a highly simplified overview of a decision tree useful in classifying arrhythmias, according to a set of rules such as PR Logic™. This process is set out in greater detail in Singer's Interventional Electrophysiology, 2^{nd} edition (June, 2001), Chapter 21 titled “Dual-Chamber Sensing and Detection for Implantable Cardioverter-Defibrillators”, by Gillberg and Olson, which chapter is herein incorporated by reference in its entirety. Each ventricular interval (i.e., an R-R interval) is a discrete unit that is evaluated and at block 100, a determination is made that there is some evidence of a possible ventricular arrhythmia. The ICD 10 evaluates the R-R intervals based upon an historic or expected value, such as a median value of a predetermined number of immediately prior cycles (e.g., 12 R-R intervals). This median R-R value is one parameter that may be used to initially trigger the ventricular arrhythmia indication (100). The ICD 10 compares (110) the median R-R interval to an SVT (supraventricular tachycardia) limit. If the median R-R interval value is under (i.e., rate is faster; thus, the interval is shorter) the SVT limit, then the ICD 10 determines that VT, FVT or VF is occurring and provides defibrillation. In other words, the ventricular rate is so high that immediate therapy is warranted and further discrimination of the arrhythmia is not provided.

Alternatively, the median R-R interval may be above the SVT limit (110) and further discrimination is permitted. The easiest type of rhythm to classify is designated as “less than” a one to one rhythm (130). That is, most of the R-R intervals are devoid of any atrial activity. This is a strong indication that the arrhythmia is VT, FVT, or VF (140) and again, therapy is delivered. The next class of rhythms is designated as 1:1 (150). That is, most R-R intervals include a single atrial event. The ICD 10 performs a pattern analysis algorithm (160) based on the PR interval and is able to discriminate between VT/VFT/VF (170), sinus tachycardia (172), or other SVTs (174). This is a highly simplified overview as a more detailed description of the PR pattern analysis (160) is not required for the present invention and various examples are provided in the material incorporated by reference. In addition, other mechanisms for discriminating 1:1 rhythms are available and may be employed herein.

The third class of rhythms is designated as having a greater than 1:1 correlation. That is, most of the R-R intervals have more than 1 atrial event. This may mean, for example, that out of every 5 R-R intervals, at least three (3) of those intervals have two (2) or more atrial events. This is the most complex type of rhythm to further discriminate. The first step (180) is to determine if double tachycardia is occurring; if so, VT/FVT/VF is occurring and therapy is delivered (200). That is, if double tachycardia is present then by definition both the atria and the ventricles have tachy rhythms and the appropriate therapy for the relevant ventricular rhythm is provided (e.g., defibrillation). An important element in determining if double tachycardia is occurring is PR dissociation (182). Association or dissociation means that a given atrial event (P) is associated with a subsequent ventricular event (R). That is, that atrial event (P) conducted and initiated the ventricular event (R). If there is PR dissociation, then the ventricular depolarization is occurring independently from the atrial event. As a generalization, if there is PR dissociation this indicates VT/FVT/VF and if there is PR association then the event is likely SVT. Again, this description is grossly simplified and discrimination criteria other than PR dissociation (182) are used to fully determine double tachycardia (180). These criteria include PR patterning, RR regularity, and a determination of the ventricular arrhythmia component (e.g., is it VF or VT).

If there is no double tachycardia (180), the next step is to determine (210) if there is ST (sinus tachycardia) or AF/AFL (atrial fibrillation/atrial flutter). The ICD 10 utilizes various discrimination criteria such as PR patterning, PP irregularity, FFRW oversensing (far field R wave), and RR regularity in making this determination. If present, then the ICD 10 may determine (220) that there is no VT/FVT/VF and withhold therapy. Conversely, if not present then this indicates that VT/FVT/VF is present (200).

Evidence of PR dissociation (182) is an important criterion in identifying double tachycardia (180), among other things. It is important to remember that each discrete R-R interval is evaluated but conclusions are typically made based upon data collected over a plurality of intervals. As indicated, a running median R-R interval is maintained. In order to be in the “greater than 1:1” decision tree a certain number of intervals must have had more than one atrial event. Thus, each R-R interval may be classified and these discreet classifications are then used in combination.

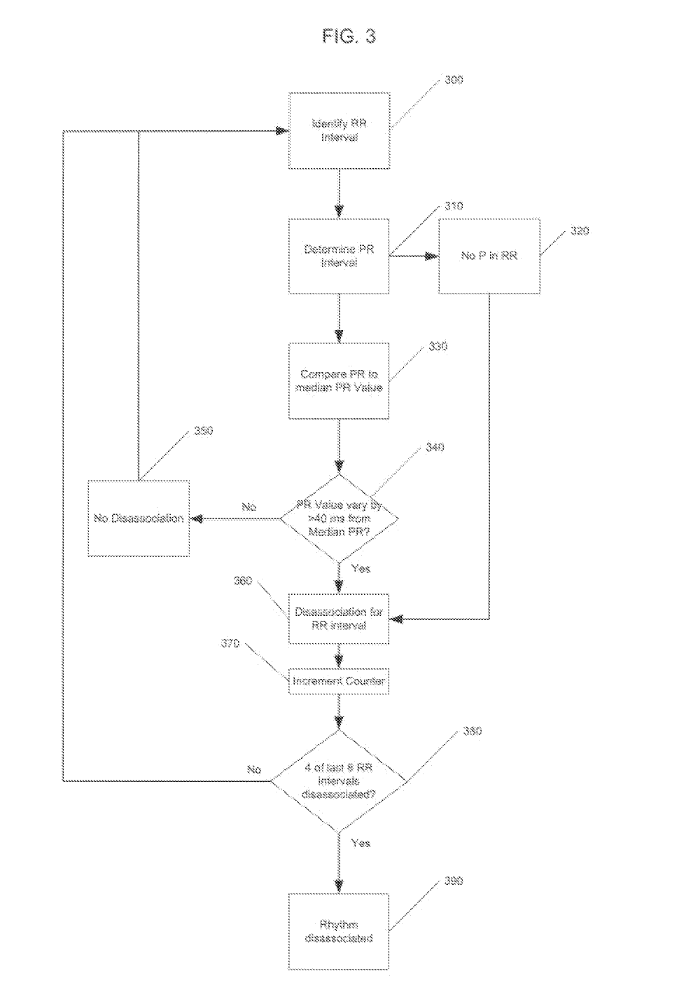

FIG. 3 is a flowchart describing a process of determining if a given R-R interval is dissociated and if an overall rhythm is dissociated. The process begins (300) with an identification of an R-R interval. Then the PR interval is determined (310). This is the time interval from the last atrial event within the R-R interval to the second R event. While most events will have multiple atrial events, there may be R-R intervals that have no atrial (P) events (320). If this is the case, then this particular cycle is classified as dissociated (360).

Assuming there was a PR interval, this interval is compared with an historic or target PR value, such as a median PR value (330). The median PR interval is based upon a predetermined number of cycles, such as the last 12 R-R intervals. The comparison (340) includes determining a difference between the current PR interval and the median PR interval. If the absolute value of this difference exceeds some predetermined quantity (e.g., 40 ms) then the current R-R cycle is classified as dissociated (360). Conversely, if the current PR interval is within 40 ms of the median, then the R-R interval is not classified as dissociated (350) and the process continues. The median value is updated accordingly.

Returning to step (360), if the R-R interval is classified as dissociated then a counter is incremented (370) and the ICD 10 determines (380) if the criteria is met to classify the rhythm as dissociated. In this example, the criteria are whether 4 of the last 8 R-R intervals were each individually dissociated. If not, the process returns to step (300), if so then the ICD 10 classifies (390) the rhythm as being dissociated. This parameter (positive or negative) is used, for example, in the arrhythmia discrimination as described in FIG. 2 where PR dissociation is relevant.

This determination of PR dissociation is generally useful; however, there are certain situations where incorrect results are returned which could lead to inappropriate therapies being delivered. It should be noted that when an error is made, it is on the side of caution with the delivery of therapy as opposed to withholding therapy. That said, it would be preferable to avoid unnecessary shocks.

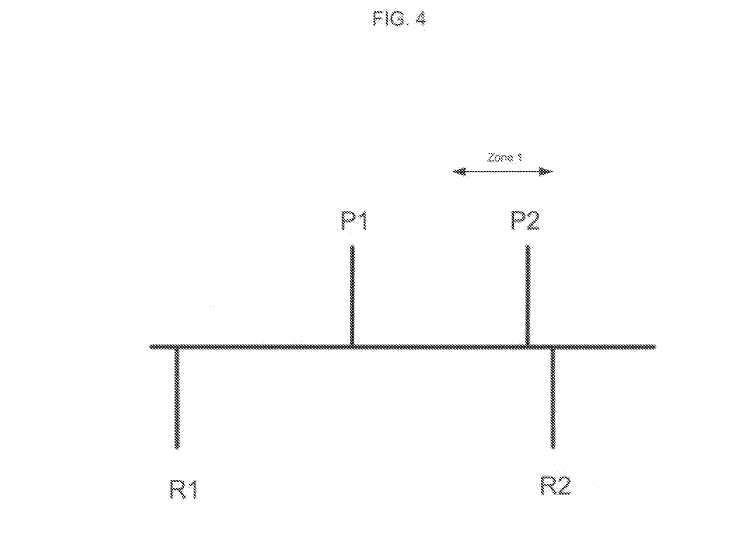

FIG. 4 illustrates a given R-R interval (R1-R2). There are two atrial events P1 (penultimate), P2 (final). In the process described in FIG. 3, the PR interval would be P2-R2 and would likely be dissociated from a median PR interval. P2 is the final atrial event that precedes in time the ventricular event R2; however, P2 is not clinically meaningful with respect to R2 because it is too close in time to have conducted. That is, the present invention provides for a consideration zone or consideration value, which in one embodiment is 60 ms. It should be appreciated that the numerical value may be varied while remaining within the scope of the present invention. Atrial events within the consideration zone are given further scrutiny in the dissociation determination.

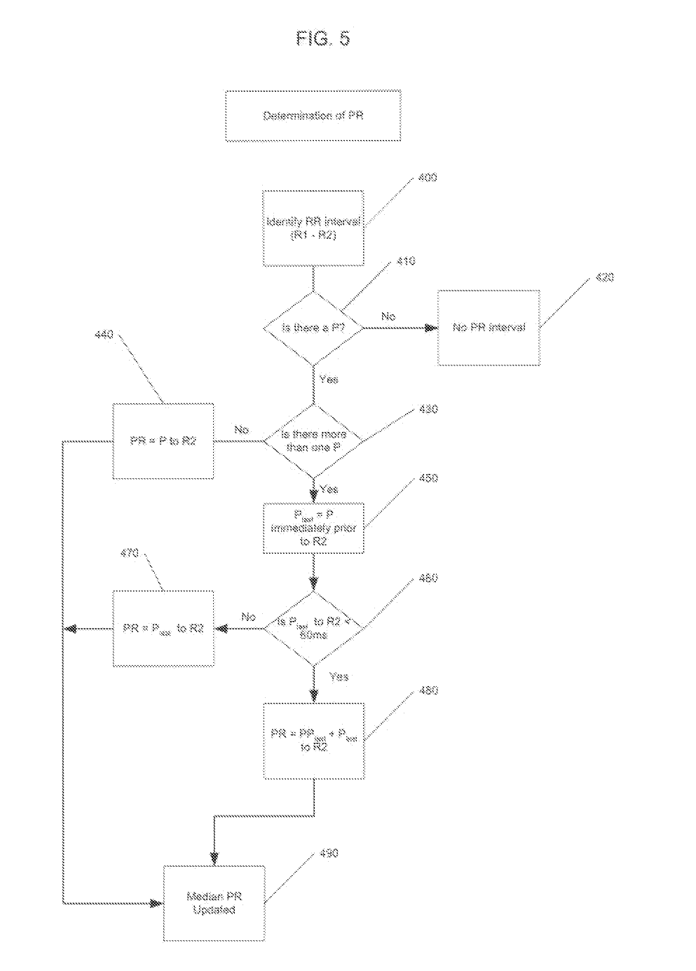

FIG. 5 is a flowchart describing a method of determining a PR interval that is then utilized in the dissociation process of FIG. 3. Once again, the individual R-R interval (R1-R2) is identified (400). The ICD 10 determines (410) if there was an atrial event (P) in the R1-R2 interval. If not (420), there is no PR interval and the RR interval is dissociated (FIG. 3). If there was an atrial event (410), the ICD 10 determines if there was more than one atrial event P within the same R1-R2 interval. If not, then the PR interval is equal to P to R2 (regardless of whether P is within the consideration zone).

If there is more then one atrial event, the one immediately prior to R2 is designated (450) as Pₗₐₛₜ. Then, the interval from Pₗₐₛₜ to R2 is calculated and if greater than, e.g., 60 ms this value is used as the PR interval for R1-R2. That is, if Pₗₐₛₜ is outside the consideration zone (e.g., 60 ms), the ICD 10 uses this value to determine the PR interval. If the interval from Pₗₐₛₜ to R2 is less than or equal to 60 ms, then the penultimate atrial event prior to R2 and therefore logically immediately prior to Pₗₐₛₜ is identified (P_{second}) and the interval between the two is designated as: P_{second}-Pₗₐₛₜ. The PR interval for R1-R2 is set (480) as:

PR_{(R1 to R2)}=((P_{second} to Pₗₐₛₜ)+(Pₗₐₛₜ to R2));

or equivalently

PR_{(R1 to R2)}=P_{second} to R2.

From either step (470) or (480), the median PR interval is updated and the determined PR interval for R1-R2 is used in the process of FIG. 3. It should be appreciated that in an alternative embodiment, the ICD 10 may determine the PR value immediately upon first evaluating the R-R interval if the immediately prior atrial event (as compared with R2) is outside of the consideration zone, without further analysis of other atrial events that may have occurred in the same R-R interval.

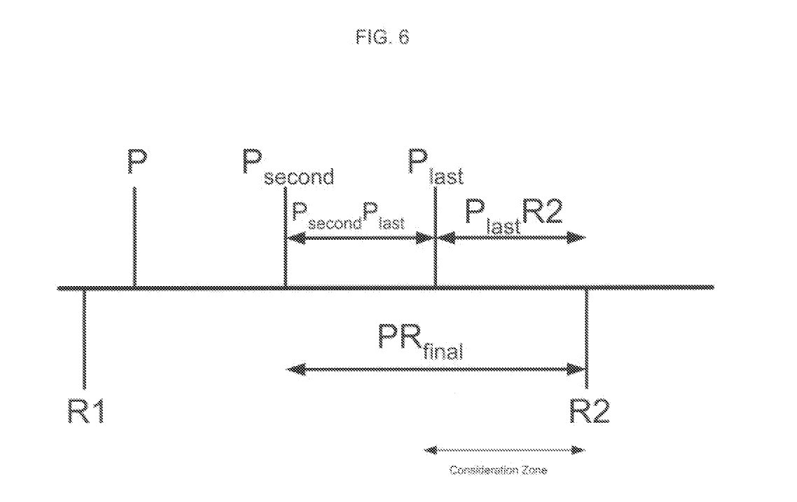

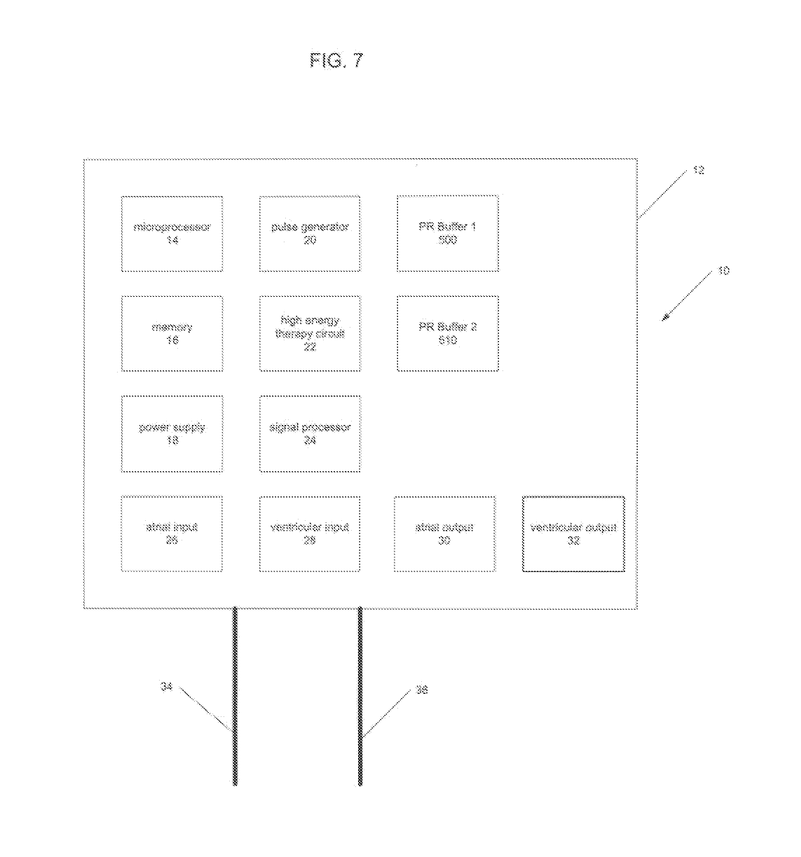

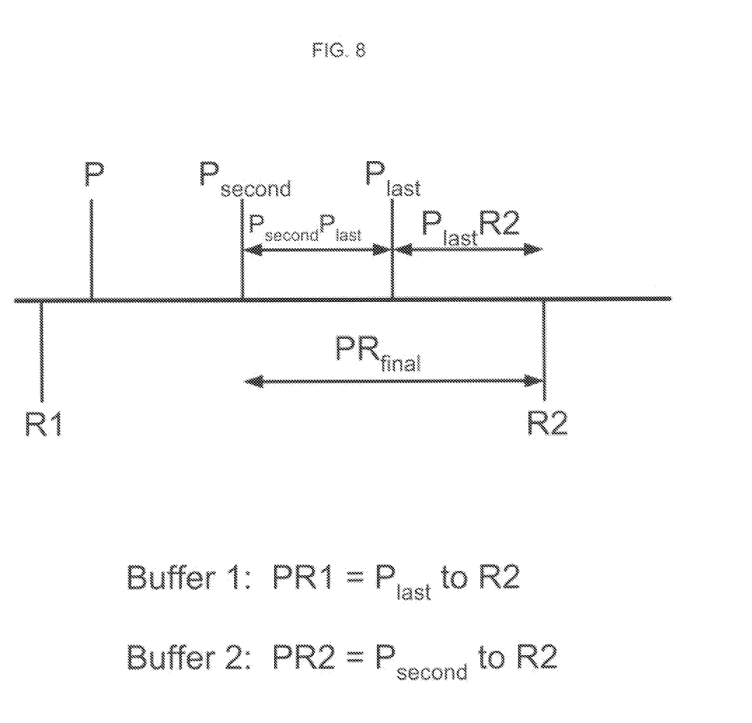

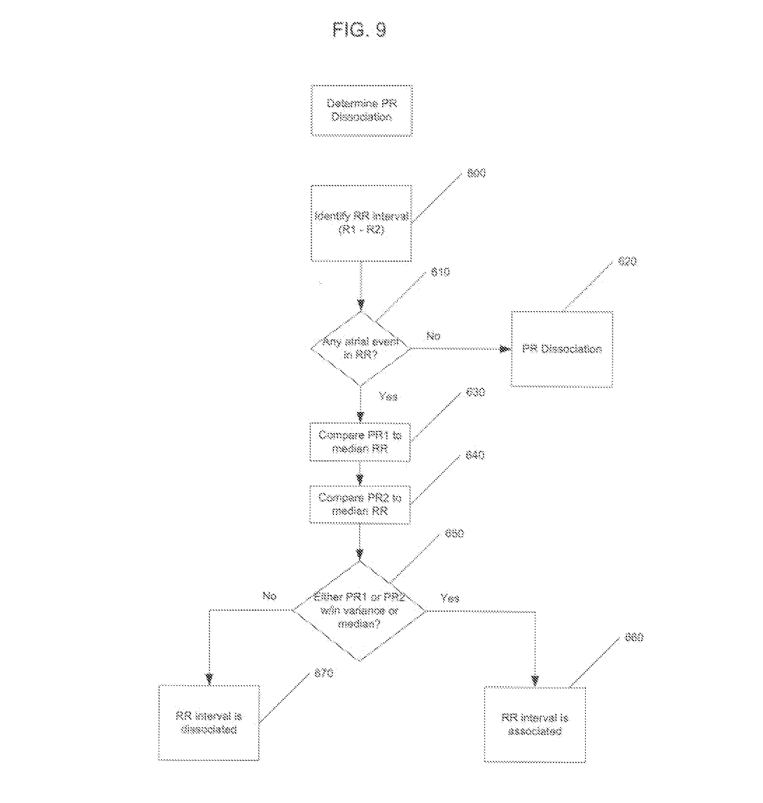

FIG. 6 is a timing diagram that graphically illustrates an example of the above with R1-R2 having multiple atrial events and Pₗₐₛₜ being within the consideration zone (e.g., 60 ms) thus, leading to the consideration of P_{second}. PR_{final} therefore becomes P_{second}—R2 which is also the same as ((P_{second} to Pₗₐₛₜ)+(Pₗₐₛₜ to R2)). It should be appreciated that even more atrial events may occur in R1-R2, as illustrated, however only the two prior to R2 are generally utilized. In one optional embodiment, if more that two atrial events are sensed and/or present within the consideration zone, then P_{second} will be the latest occurring atrial event outside the consideration zone.

When Pₗₐₛₜ is within the consideration zone, it means that it was unlikely to have conducted and result in the ventricular depolarization R2. If it was the sole atrial event, the conclusion drawn of dissociation is therefore accurate. However, when there is a second (earlier) atrial event P_{second} it is quite possible that this event conducted and resulted in R2. Thus, this is a more accurate PR interval to consider in this R-R interval. Ultimately, if neither resulting PR interval is within, e.g., 40 ms of the median PR value, dissociation will be indicated; however, the number of falsely indicated dissociated events will be reduced as compared with a determination based solely upon the last atrial event in a given R-R interval. As dissociation is an indicator of VT/FVT/VF, improperly classifying an interval as dissociated may contribute to unnecessary shocks and conversely, improving the accuracy of dissociation determinations will reduce the number of unnecessary shock thereby improving patient satisfaction with the implantable medical device.

FIG. 7 illustrates another embodiment of ICD 10 consistent with the teachings of the present invention. In this embodiment, ICD 10 includes two separate PR buffers. Specifically, (1) PR Buffer 500 and (2) PR Buffer 510. These two PR buffers 500, 510 will run simultaneously for each RR interval, recording data as will be described. As these are two distinct, parallel components they may be provided as such in the hardware or firmware of the ICD 10.

FIG. 8 is a timing diagram that illustrates what data is provided in each of the two buffers. Here PR Buffer 1 is the interval from the last atrial event in the RR interval to the end of the RR interval, or Pₗₐₛₜ to R2. The data in the PR Buffer 2 is the interval from the penultimate atrial event to the end of the RR interval, or P_{second} to R2. It is not important which particular buffer includes a specific interval.

In practice, a first sensed atrial event (P in FIG. 8), initiates the interval stored in e.g., PR Buffer 1. When P_{second} occurs, an interval begins and is stored in PR Buffer 2. Then, when Pₗₐₛₜ occurs, the PR Buffer 1 is cleared and reset to begin with Pₗₐₛₜ, while PR Buffer 2 continues. The end of the RR interval (R2) is the end of point for each of the intervals in PR Buffers 1 & 2. In other words, the parallel buffers 500, 510 will record the last PR interval and the second to last PR interval in any given RR interval and if there are more atrial events, only the last two remain relevant as the earlier events are overwritten. Of course, in alternative embodiments, three or more buffers may be provided.

FIG. 9 is a flowchart that describes the process of determining PR dissociation, according to with the present embodiment. The ICD 10 identifies (600) the relevant RR interval. As previously described, a determination is made (610) as to whether any atrial event occurred during the RR interval. If not, then the interval is classified as dissociated (620).

Assuming there was at least one atrial event, then the ICD 10 compares (630, 640) the intervals in PR Buffer 1 and PR Buffer 2 to the median PR interval. This assumes that a sufficient data set has been acquired to determine the median value. If either the value of PR Buffer 1 or 2 is within (650) the predefined variance (e.g., 40 ms) of the median PR value, then the RR interval is classified as associated (660). Conversely, if neither the value of PR Buffer 1 nor 2 is within the predefined variance of the median PR value, then the RR interval is classified as dissociated (670). If only one atrial event occurs, then only one buffer is required. The second buffer may remain at a null point or may parallel the first buffer so that they each have the same value, if this functionality is preferred. By capturing the last PR value and the second to last PR value with the dual buffer arrangement, the ICD 10 is not forced solely to evaluate the last atrial event and as a practical matter, capturing these two values should have an accuracy in classifying dissociation approximating that when using the consideration zone.

In each of the embodiments described herein, a given RR interval is determined to be associated or dissociated based on an absence of an atrial event or an appropriate PR interval if atrial event(s) are sensed. If only one atrial event is sensed, then the PR interval is set by default to that value. If more than one atrial event occurs, the present invention provides various mechanisms to utilize an appropriate value. More specifically, to avoid only having the last PR interval available and having to use that value by default. As described earlier, a consideration zone may be provided and if an atrial event occurs within this window, the ICD 10 will utilize an alternative atrial event outside the window, if available. In other embodiments, the consideration zone is not utilized in lieu of dual PR buffers. As a practical matter, none of the potentially available PR intervals will lead to a classification of association unless that PR interval is within a predetermined variance of an established median PR value. Finally, in other embodiments, multiple PR buffers (two or more) may be utilized along with a consideration zone.

While various embodiments have been shown and described it should be appreciated that one of ordinary skill in the art would recognize various variations of these embodiments as being within the scope and spirit of the present invention. Further, various combinations of elements and embodiments may be made, though not specifically illustrated while also remaining within the spirit and scope of the present invention.

1. A method of determining PR dissociation comprising:
generating a target PR value from a plurality of preceding R-R intervals;
identifying a given R-R interval;
running a first buffer to capture a last PR interval within the R-R interval, if present in the R-R interval;
running a second buffer to capture a penultimate PR interval within the R—R interval, if present within the R-R interval;
comparing the last PR interval from the first buffer and the penultimate PR interval from the second buffer to the target PR value; and
determining if the current RR interval is dissociated based upon the comparison.

2. The method of claim 1, further comprising updating the target PR value with the current PR value for an R-R interval subsequent to the given R-R interval.

3. The method of claim 1, wherein the target PR value is a median of PR intervals from a predetermined number of RR intervals.

4. The method of claim 1, wherein the determination indicates dissociation if neither the last PR interval nor the penultimate PR interval is within a predetermined variance of the target PR value.

5. The method of claim 1, wherein comparing includes obtaining an absolute value of a difference between the target PR value and each of the last PR interval and the penultimate PR interval and determining further includes indicating dissociation if the absolute value of the difference for each exceeds a first predetermined value.

6. The method of claim 5, wherein the first predetermined values is approximately 40 ms.

7. The method of claim 1, further comprising:
determining if there were no atrial events, a single atrial event, or multiple atrial events within the given R-R interval;
classifying the given R-R interval as dissociated if there were no atrial events within the given R-R interval; and
setting the current PR interval equal to the duration between the single atrial event and the end of the given R-R interval.

8. The method of claim 1, further comprising:
detecting a greater than 1:1 tachyarrhythmia; and
utilizing the determination of dissociation in discriminating between atrial and ventricular arrhythmia.

9. The method of claim 8, wherein discriminating between atrial and ventricular arrhythmias includes identifying whether or not a double tachyarrhythmia is occurring.

10. An implantable medical device (IMD) comprising:
means for generating a target PR value from a plurality of preceding R-R intervals;
means for identifying a given R-R interval;
means for running a first buffer to capture a last PR interval within the R-R interval, if present in the R-R interval;
means for running a second buffer to capture a penultimate PR interval within the R-R interval, if present within the R-R interval;
means for comparing the last PR interval from the first buffer and the penultimate PR interval from the second buffer to the target PR value; and
means for determining if the current RR interval is dissociated based upon the comparison.

11. The IMD of claim 10, wherein the means for comparing further include means for obtaining an absolute value of a difference between the target PR value and each of the last PR interval and the penultimate PR interval and indicating dissociation if the absolute value of the difference for each exceeds a first predetermined value.

12. The IMD of claim 10, further comprising:
means for determining if there were no atrial events, a single atrial event, or multiple atrial events within the given R-R interval;
means for classifying the given R-R interval as dissociated if there were no atrial events within the given R-R interval; and
means for setting the current PR interval equal to the duration between the single atrial event and the end of the given R-R interval.

13. A computer readable medium comprising instructions that when executed on a processor of an implantable medical device (IMD), cause the IMD to:
generate a target PR value from a plurality of preceding R-R intervals;
identify a given R-R interval;
operate a first buffer to capture a last PR interval within the R-R interval, if present in the R-R interval;
operate a second buffer to capture a penultimate PR interval within the R—R interval, if present within the R-R interval;
comparing the last PR interval from the first buffer and the penultimate PR interval from the second buffer to the target PR value; and
determining if the current RR interval is dissociated based upon the comparison.

14. The computer readable medium of claim 13, wherein the instructions further cause the IMD to update the target PR value with the current PR value selected from the first buffer or from the second buffer for an R-R interval subsequent to the given R-R interval.

15. The computer readable medium of claim 13, wherein the target PR value is a median of PR intervals from a predetermined number of RR intervals.

16. The computer readable medium of claim 13, wherein when the instructions cause the IMD to compare, the IMD further obtains an absolute value of a difference between the target PR value and each of the last PR interval and the penultimate PR interval and indicates dissociation if the absolute value of the difference for each exceeds a first predetermined value.

17. The computer readable medium of claim 16, wherein the first predetermined value is approximately 40 ms.

18. The computer readable medium of claim 13, wherein the instructions further cause the IMD to:
determine if there were no atrial events, a single atrial event, or multiple atrial events within the given R-R interval;
classify the given R-R interval as dissociated if there were no atrial events within the given R-R interval; and
set the current PR interval equal to the duration between the single atrial event and the end of the given R-R interval.
